(19) United States
(12) Patent Application Publication
(10) Pub. No.: US 2011/0238060 A1
(43) Pub. Date: Sep. 29, 2011

(54) MICROWAVE SURGICAL DEVICE

(75) Inventors: Fred T. Lee, JR., Madison, WI (US); Christopher L. Brace, Middleton, WI (US); Paul F. Laeseke, Madison, WI (US); Daniel Warren van der Weide, Madison, WI (US)

(73) Assignee: NEUWAVE MEDICAL, INC., Madison, WI (US)

(21) Appl. No.: 13/153,974

(22) Filed: Jun. 6, 2011

Related U.S. Application Data
(63) Continuation of application No. 11/440,331, filed on May 24, 2006.
Application No. 11/440,331 is a continuation-in-part of application No. 10/834,802, filed on Apr. 29, 2004, now Pat. No. 7,101,369.

(51) Int. Cl. A61B 18/04

(52) U.S. Cl. 606/33

(57) ABSTRACT

A medical instrument or device used to decrease blood loss during surgery and/or other medical procedures. The device includes a microwave antenna housed in a handset (or laparoscopic probe) that is placed in close proximity to the tissue of interest. The device runs in the microwave spectrum and receives power from a from a microwave generator. When turned on (triggered), the device delivers microwave energy to tissue, providing a cutting or cautery effect.

### CROSS-REFERENCE TO RELATED APPLICATIONS

This application is a Continuation of pending U.S. patent application Ser. No. 11/440,331, filed May 24, 2006, which is a Continuation-in-Part of pending U.S. patent application Ser. No. 10/834,802, filed Apr. 29, 2004 (now U.S. Pat. No. 7,101,369, issued Sep. 5, 2006), which claims priority to expired U.S. Provisional Patent Application No. 60/684,065, filed May 24, 2005, and to expired U.S. Provisional Patent Application No. 60/690,370, filed Jun. 14, 2005, and to expired U.S. Provisional Patent Application No. 60/702,393, filed Jul. 25, 2005, and to expired U.S. Provisional Patent Application No. 60/707,797, filed Aug. 12, 2005, and to expired U.S. Provisional Patent Application No. 60/710,276, filed Aug. 22, 2005, and to expired U.S. Provisional Patent Application No. 60/710,815, filed Aug. 24, 2005, the contents of which are incorporated herein by reference in their entireties.

### FIELD OF INVENTION

The present disclosure relates to medical instruments for decreasing blood loss, and assisting in tissue cutting during surgery and/or other medical procedures.

### BACKGROUND

Blood loss during surgery is a substantial clinical problem. Resection of multiple tissue types in the neck, chest, abdomen, pelvis, and extremities are associated with blood loss that can be acutely life-threatening from hemodynamic effects, or if the blood loss is severe enough, can require transfusions. This can be problematic from an immunological point of view during cancer surgery. For example, increased blood loss requiring transfusions during hepatic resection increases post-resection mortality. Blood loss is also a major problem during surgery for sharp or blunt trauma, in orthopedic surgery, and in gynecologic and obstetrical procedures.

Current electrosurgical devices used for cautery and cutting, discussed below, have various associated problems and disadvantages as are known in the art. Accordingly, there is a need for a device which decreases blood loss during surgery, which overcomes the problems and disadvantages associated with current electrosurgical devices used for cautery and cutting, and which is an improvement thereover.

### SUMMARY

The device of the present disclosure is a microwave device that can be used to decrease blood loss during surgery. This device is different than electrocautery devices based on radiofrequency that are in widespread clinical use. The microwave surgical device described in this disclosure is comprised of a microwave antenna housed in a handset (or laparoscopic probe) that is placed in close proximity to the tissue of interest. When turned on (triggered), the device delivers microwave energy to tissue, providing a cautery or cutting, or combined cautery and cutting effect. Tissue can then be divided rapidly and without fear of untoward hemorrhage. This device can also be used to stop pre-existing hemorrhage on a small or large scale. For example, during open abdominal procedures, a small blood vessel can be near instantaneously cauterized by applying microwave energy directly to it.

The present invention provides a triaxial microwave probe design for MWA where the outer conductor allows improved tuning of the antenna to reduce reflected energy through the feeder line. This improved tuning reduces heating of the feeder line allowing more power to be applied to the tissue and/or a smaller feed line to be used. Further, the outer conductor may slide with respect to the inner conductors to permit adjustment of the tuning in vivo to correct for effects of the tissue on the tuning.

Specifically, the present invention provides a probe for microwave ablation having a first conductor and a tubular second conductor coaxially around the first conductor but insulated therefrom. A tubular third conductor is fit coaxially around the first and second conductors. The first conductor may extend beyond the second conductor into tissue when a proximal end of the probe is inserted into a body for microwave ablation. The second conductor may extend beyond the third conductor into the tissue to provide improved tuning of the probe limiting power dissipated in the probe outside of the exposed portions of the first and second conductors.

Thus, it is one object of at least one embodiment of the invention to provide improved tuning of an MWA device to provide greater power to a lesion without risking damage to the feed line or burning of tissue about the feed line and/or to allow smaller feed lines in microwave ablation.

The third tubular conductor may be a needle for insertion into the body. The needle may have a sharpened tip and may use an introducer to help insert it.

Thus, it is another object of at least one embodiment of the invention to provide a MWA probe that may make use of normal needle insertion techniques for placement of the probe.

It is another object of at least one embodiment of the invention to provide a rigid outer conductor that may support a standard coaxial for direct insertion into the body.

The first and second conductors may fit slidably within the third conductor.

It is another object of at least one embodiment of the invention to provide a probe that facilitates tuning of the probe in tissue by sliding the first and second conductors inside of a separate introducer needle.

The probe may include a lock attached to the third conductor to adjustably lock a sliding location of the first and second conductors with respect to the third conductor.

It is thus another object of at least one embodiment of the invention to allow locking of the probe once tuning is complete.

The probe may include a stop attached to the first and second conductors to about a second stop attached to the third conductor to set an amount the second conductor extends beyond the tubular third conductor into tissue. The stop may be adjustable.

Thus, it is another object of at least one embodiment of the invention to provide a method of rapidly setting the probe that allows for tuning after a coarse setting is obtained.

The second conductor may extend beyond the third conductor by an amount L 1 and the first conductor may extend beyond the second conductor by an amount L 2 and L 1 and L 2 may be multiples of a quarter wavelength of a microwave frequency received by the probe.

It is thus another object of at least one embodiment to promote a standing wave at an antenna portion of the probe.

These particular objects and advantages may apply to only some embodiments falling within the claims and thus do not define the scope of the invention.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1A is a chart illustrating the dependence of the coagulation diameter on the length of time of use of the device of the present disclosure.

FIG. 1B is a chart illustrating the dependence of the coagulation diameter on the amount of applied power during use of the device of the present disclosure.

FIG. 2 is a diagram of a delivery tool and control/feedback system for cauterizing tissue, illustrating a preferred embodiment of the present disclosure.

FIG. 3 is a schematic, cross-sectional diagram of an embodiment of an antenna and scalpel combination of the present disclosure.

FIG. 4 is a schematic diagram of an embodiment of an antenna and scissors combination of the present disclosure.

FIG. 5 is a schematic representation of a microwave power supply attached to a probe of the present invention for percutaneous delivery of microwave energy to a necrosis zone within an organ.

FIG. 6 is a perspective fragmentary view of the proximal end of the probe of FIG. 5 showing exposed portions of a first and second conductor slideably received by a third conductor and showing a sharpened introducer used for placement of the third conductor.

FIG. 7 is a fragmentary cross sectional view of the probe of FIG. 6 showing connection of the microwave power supply to the first and second conductors.

FIG. 8 is a cross sectional view of an alternative embodiment of the probe showing a distal electric connector plus an adjustable stop thumb screw and lock for tuning the probe.

### DESCRIPTION OF DISCLOSED EMBODIMENT

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

The device of the present disclosure is different than current electrosurgical devices that are used for cautery and cutting. The disclosed device will run in the microwave (not radiofrequency) spectrum and receives power from a from a microwave generator. The preferred frequencies would be the ISM (Industrial, Scientific and Medical) bands at 915 MHz, 2.45 GHz, and 5.8 GHz, although other frequencies could also be used. Since the device is not radiofrequency based, there is no need for ground pads, and charring will not substantially affect the ability of this device to perform a cautery or cut function.

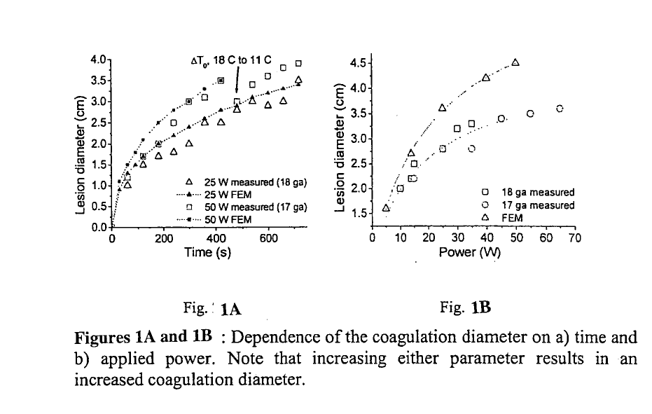

The depth of penetration of the coagulation effect can be varied depending on the amount of power that is applied, the angle at which the device is held, and the duration that the device is held in proximity to the tissue. For example, experimental data show that a region greater than 2 cm in diameter can be coagulated in 2 minutes with an input power of ˜65 W. Data also shows the ablation zone diameter may be controlled by varying input power and application time (FIGS. 1A and 1B).

The specific antenna design can be variable. One possibility is to construct the microwave delivery tool based on a triaxial design, thereby taking advantage of the resonant frequency effects of triaxial catheters. However, many microwave delivery systems (e.g. coaxial near-field antennas) can be used for this purpose if they are designed to have a short protrusion of the center conductor (e.g. protrusion approximately the radius of the coaxial cable) such that in near-contact with tissue, a large absorption of microwave power is achieved.

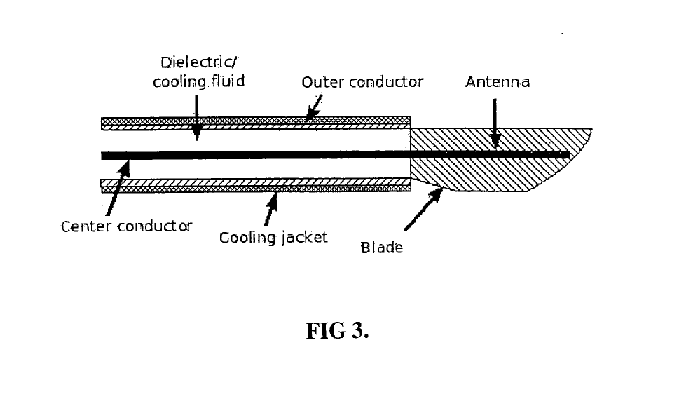

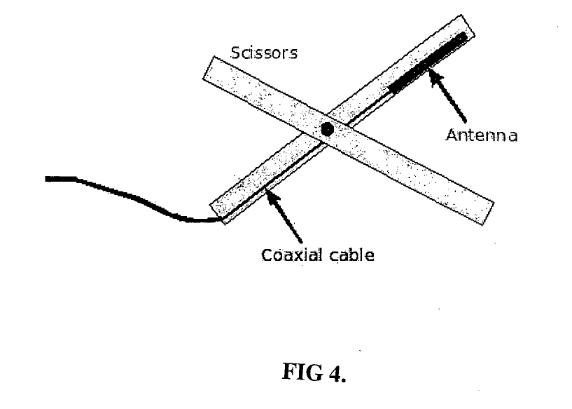

Other antenna designs may include dielectric resonators, particularly those formed n the shape of a mechanical cutting tool; coplanar, microstrip or similar waveguiding and radiating structures; spiral or helical antennas with the helix axis parallel to the coaxial feed line; planar spiral antennas; two-sided balanced or unbalanced transmission lines; antennas mounted as part of a scissors (FIG. 4), knife or scalpel (FIG. 3), clamp or other cutting or pressure-inducing device. Experiments conducted during the development of embodiments of the present invention illustrated various cuts and coagulation of porcine liver tissue created by the device of the present disclosure using a coaxial monopole antenna.

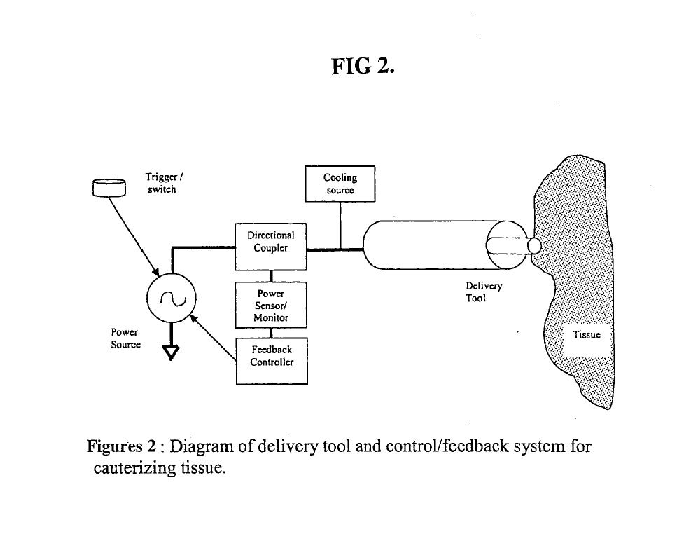

As shown in FIG. 2, the system may deliver power to the tool through a trigger switch, foot pedal or other switch or on/off button. Power reflected from the antenna can be detected and monitored to provide feedback for power control or as a safety interlock to interrupt the microwave power source if the reflected power exceeds a threshold. The control and feedback loop varies the power or duty cycle of the microwave source, enabling both safe operation and variable power application. Further, the tool can have an adjustment or calibration mechanism wherein the device can be tuned relative to the tissue of interest to a low reflected power prior to use.

The device can be mounted in a handle that is cooled by circulating fluid, gas or liquid metal. In addition, cooling fluid, gas, or liquid metal can be circulated through the center of the antenna to reduce untoward line heating as well as vary the characteristic impedance of the antenna. In one embodiment, the antenna operates at a preferential frequency of 77Ω. to reduce line heating. Alternatively or in addition, the antenna can have an air-core or vacuum-core design to reduce dielectric heating. The feed of the antenna can be comprised of any conductive metal including copper, stainless steel or titanium, and the shaft can be insulated with various thermal insulators such as parylene or Teflon. The delivery tool can be coated with a biocompatible coating (e.g. a polymer such as Paralyne), and can be cooled with a water jacket.

As stated previously, this device could be used at conventional open surgery, laparoscopy, and/or percutaneously for the purpose of coagulation, vessel sealing, or cutting. The application end could house a mechanical scalpel or any other type of device to divide tissue to make an “all in one” coagulation and cutting device. The antenna could be mounted in combination with other surgical tools (one example is with a conventional scalpel), scissors, or used as a needle to stop hemorrhage. The depth of electromagnetic field penetration could be varied depending on the particular use; for example in neurosurgery, a very small amount of penetration would be desirable.

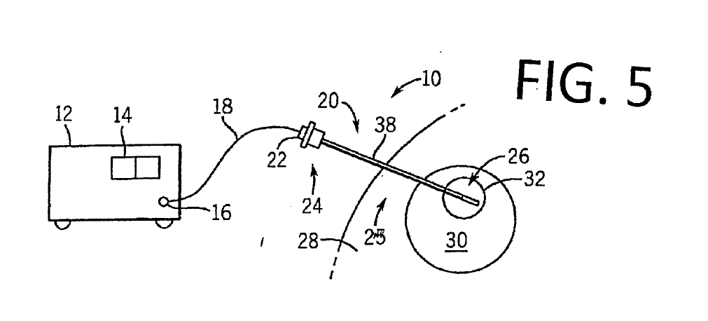

Referring now to FIG. 5, a microwave ablation device 10 per the present invention includes a microwave power supply 12 having an output jack 16 connected to a flexible coaxial cable 18 of a type well known in the art. The cable 18 may in turn connect to a probe 20 via a connector 22 at a distal end 24 of the probe 20.

The probe 20 provides a shaft 38 supporting at a proximal end 25 an antenna portion 26 which may be inserted percutaneously into a patient 28 to an ablation site 32 in an organ 30 such as the liver or the like.

The microwave power supply 12 may provide a standing wave or reflected power meter 14 or the like and in the preferred embodiment may provide as much as 100 watts of microwave power of a frequency of 2.45 GHz. Such microwave power supplies are available from a wide variety of commercial sources including as Cober-Muegge, LLC of Norwalk, Conn., USA.

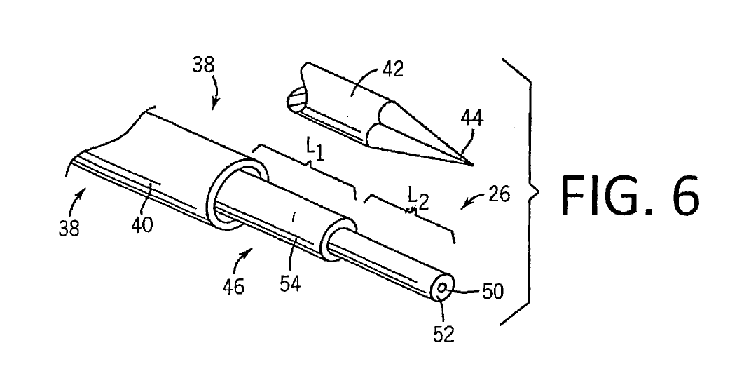

Referring now to FIGS. 5 and 6, generally a shaft 38 of the probe 20 includes an electrically conductive tubular needle 40 being, for example, an 18-gauge needle of suitable length to penetrate the patient 28 to the ablation site 32 maintaining a distal end 24 outside of the patient 28 for manipulation.

Either an introducer 42 or a coaxial conductor 46 may fit within the needle 40. The introducer 42 may be a sharpened rod of a type well known in the art that plugs the opening of the needle 40 and provides a point 44 facilitating the insertion of the probe 20 through tissue to the ablation site 32. The needle 40 and introducer 42 are of rigid material, for example, stainless steel, providing strength and allowing easy imaging using ultrasound or the like.

The coaxial conductor 46 providing a central first conductor 50 surrounded by an insulating dielectric layer 52 in turn surrounded by a second outer coaxial shield 54. This outer shield 54 may be surrounded by an outer insulating dielectric not shown in FIG. 6 or may be received directly into the needle 40 with only an insulating air gap between the two. The coaxial conductor 46 may, for example, be a low loss 0.86-millimeter coaxial cable.

Referring still to FIG. 6, the central conductor 50 with or without the dielectric layer 52, extends a distance L 2 out from the conductor of the shield 54 whereas the shield 54 extends a distance L 1 out from the conductor of the needle 40. L 1 is adjusted to be an odd multiple of one quarter of the wavelength of the frequency of the microwave energy from the power supply 12. Thus the central conductor 50 in the region of L 2 provides a resonant monopole antenna having a peak electrical field at its proximal end and a minimal electric field at the end of the shield 54 as indicated by 56.

At 2.45 GHz, the length L 2 could be as little as 4.66 millimeters. Preferably, however, a higher multiple is used, for example, three times the quarter wavelength of the microwave power making L 2 approximately fourteen millimeters in length. This length may be further increased by multiple half wavelengths, if needed.

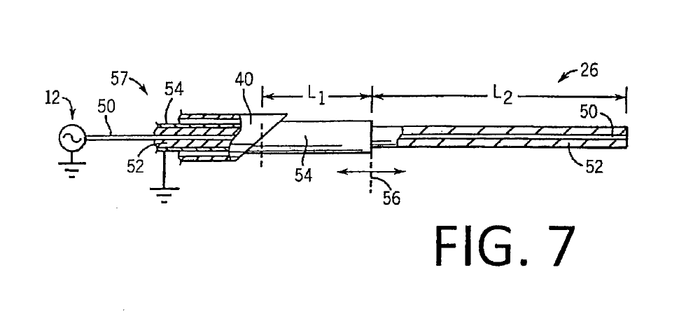

Referring to FIG. 7, the length L 1 is also selected to be an odd multiple of one quarter of the wavelength of the frequency of the microwave energy from the power supply 12. When needle 40 has a sharpened or bevel cut tip, distance L 1 is the average distance along the axis of the needle 40 of the tip of needle 40.

The purpose of L 1 is to enforce a zero electrical field boundary condition at line 56 and to match the feeder line 56 being a continuation of coaxial conductor 46 within the needle 40 to that of the antenna portion 26. This significantly reduces reflected energy from the antenna portion 26 into the feeder line 56 preventing the formation of standing waves which can create hot spots of high current. In the preferred embodiment, L 1 equals L 2 which is approximately fourteen millimeters.

The inventors have determined that the needle 40 need not be electrically connected to the power supply 12 or to the shield 54 other than by capacitive or inductive coupling. On the other hand, small amounts of ohmic contact between shield 54 and needle 40 may be tolerated.

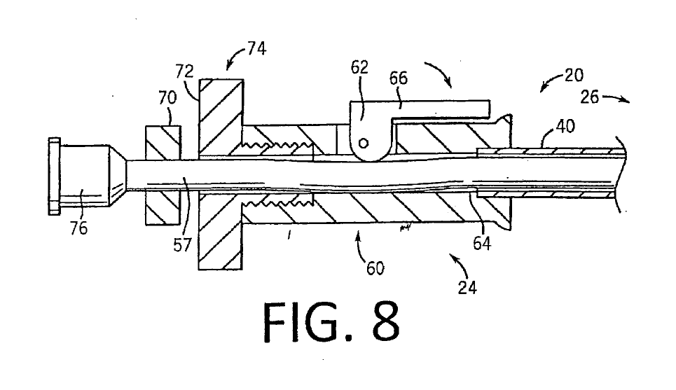

Referring now to FIGS. 5, 6 and 8, during use, the combination of the needle 40 and introducer 42 are inserted into the patient 28, and then the introducer 42 is withdrawn and replaced by a the coaxial conductor 46 so that the distance L 2 is roughly established. L 2 has been previously empirically for typical tissue by trimming the conductor 50 as necessary.

The distal end 24 of needle 40 may include a tuning mechanism 60 attached to the needle 40 and providing an inner channel 64 aligned with the lumen of the needle 40. The tuning mechanism provides at its distal end, a thumbwheel 72 having a threaded portion received by corresponding threads in a housing of the tuning mechanism and an outer knurled surface 74. A distal face of the thumbwheel provides a stop that may abut a second stop 70 being clamped to the coaxial conductor 46 thread through the tuning mechanism 60 and needle 40. When the stops 70 and on thumbwheel 72 abut each other, the coaxial conductor 46 will be approximately at the right location to provide for extension L 1. Rotation of the thumbwheel 72 allows further retraction of the coaxial conductor 46 to bring the probe 20 into tuning by adjusting L 1. The tuning may be assessed by observing the reflected power meter 14 of FIG. 5 and tuning for reduced reflected energy.

The tuning mechanism 60 further provides a cam 62 adjacent to the inner channel 64 through which the coaxial conductor 46 may pass so that the cam 62 may press and hold the coaxial conductor 46 against the inner surface of the channel 64 when a cam lever 66 is pressed downwards 68. Thus, once L 1 is properly tuned, the coaxial conductor 46 may be locked in position with respect to needle 40.

The distal end of the coaxial conductor 46 may be attached to an electrical connector 76 allowing the cable 18 to be removably attached to disposable probes 20.

The present invention provides as much as a ten-decibel decrease in reflected energy over a simple coaxial monopole in simulation experiments and can create a region of necrosis at the ablation site 32 greater than two centimeters in diameter.

It is to be understood that the embodiment(s) herein described is/are merely illustrative of the principles of the present invention. Various modifications may be made by those skilled in the art without departing from the spirit or scope of the claims which follow.

1. A device configured for cutting and/or cauterizing tissue, wherein said device comprises a blade configured for cutting tissue, wherein said device comprises a tool configured for cauterizing tissue through delivery of microwave energy to said tissue, wherein said tool comprises a triaxial antenna for delivering microwave energy, wherein said triaxial antenna is mounted with said blade, said triaxial antenna comprising:
a first conductor,
a tubular second conductor coaxially around the first conductor but insulated therefrom,
a tubular third conductor coaxially around the first and second conductors;
a tuning mechanism having a locked state fixedly holding the third conductor against axial movement with respect to the first and second conductors and having a unlocked state allowing axial movement between the third conductor and the first and second conductors;
wherein the first conductor extends beyond the second conductor into tissue, when a distal end of the probe is inserted into a body for microwave ablation, to promote microwave frequency current flow between the first and second conductors through the tissue; and
wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors.

2. A surgical device configured for cutting and/or cauterizing tissue, wherein said device comprises a blade configured for cutting tissue, wherein said device comprises a tool configured for cauterizing tissue through delivery of microwave energy to said tissue, wherein said tool comprises an antenna for delivering microwave energy, wherein said triaxial antenna is mounted with said blade, wherein the characteristic impedance for said antenna is 77 ohms, said antenna comprising:
a first conductor,
a tubular second conductor coaxially around the first conductor but insulated therefrom,
a tubular third conductor coaxially around the first and second conductors;
a tuning mechanism having a locked state fixedly holding the third conductor against axial movement with respect to the first and second conductors and having a unlocked state allowing axial movement between the third conductor and the first and second conductors;
wherein the first conductor extends beyond the second conductor into tissue, when a distal end of the probe is inserted into a body for microwave ablation, to promote microwave frequency current flow between the first and second conductors through the tissue; and
wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors.

3. The device of claim 2, wherein said device has therein a handset, wherein the microwave antenna is housed in said handset.

4. The device of claim 2, wherein the microwave antenna receives power from a microwave generator.

5. The device of claim 2, wherein the antenna has a length and an insertion depth, and wherein the length and insertion depth of the antenna are tunable.

6. The device of claim 2, wherein the antenna has a reflection coefficient, and wherein the reflection coefficient of the antenna is tunable.

7. The device of claim 2, wherein the microwave antenna is coplanar or constructed from coplanar waveguide or uses a coplanar waveguide feed.

8. The device of claim 2, wherein the microwave antenna is constructed from microstrip waveguide or uses a microstrip waveguide feed.

9. The device of claim 2, wherein the microwave antenna is constructed of balanced or unbalanced two-line transmission line.

10. The device of claim 2, wherein the microwave antenna is a dielectric resonator, having a blade or scalpel like shape.

11. The device of claim 2, wherein the microwave antenna is mounted as part of a clamp or pressure inducing device.

12. The device of claim 2, wherein the antenna includes dielectric material, and wherein the dielectric material of the coaxial delivery system is one of a fluid and a vacuum.

13. The device of claim 2, wherein at least a portion of the microwave antenna is cooled.

14. The device of claim 12, wherein the microwave antenna is configured to circulate a cooling fluid around the exterior of the microwave antenna, through a portion of the dielectric material, or through a portion of the center conductor.

15. The device of claim 2, wherein the microwave antenna is controlled through a switch mechanism.

16. The device of claim 2, wherein the microwave antenna is operatively connected to a directional coupler in combination with a power sensor and a feedback controller.

17. The device of claim 2, wherein reflected power of the microwave antenna is monitored.

18. The device of claim 17, wherein the monitored reflected power is used to control the antenna input power, application time or schedule.

19. The device of claim 17, wherein the monitored reflected power is used in an interlocking safety circuit to limit or eliminate antenna input power when a threshold reflected power is surpassed.

20. The device of claim 2, wherein said blade is a scalpel, scissors or other cutting device.

21. A surgical method, comprising the steps of:
supplying power from a microwave generator to a triaxial microwave antenna contained in a cutting device, wherein said cutting device has therein a blade, wherein said triaxial microwave antenna is mounted with said blade, said triaxial microwave antenna comprising:
a first conductor,
a tubular second conductor coaxially around the first conductor but insulated therefrom,
a tubular third conductor coaxially around the first and second conductors;
a tuning mechanism having a locked state fixedly holding the third conductor against axial movement with respect to the first and second conductors and having a unlocked state allowing axial movement between the third conductor and the first and second conductors;
wherein the first conductor extends beyond the second conductor into tissue, when a distal end of the probe is inserted into a body for microwave ablation, to promote microwave frequency current flow between the first and second conductors through the tissue; and
wherein the second conductor may be adjusted by the tuning mechanism to extend beyond the third conductor into tissue when an end of the probe is inserted into the body for microwave ablation to provide improved tuning of the probe limiting power dissipated in the probe outside of exposed portions of the first and second conductors; and
placing the triaxial microwave antenna in close proximity to tissue of interest such that the tissue of interest is cauterized.

22. The method of claim 21, wherein said cutting device is selected from the group consisting of a scalpel and scissors.

23. The method of claim 21, wherein the characteristic impedance for the triaxial microwave antenna is 77 ohms.
